(19) United States
(12) Patent Application Publication
(10) Pub. No.: US 2024/0350087 A1
(43) Pub. Date: Oct. 24, 2024

(54) PROXIMITY SENSOR IN PULSE OXIMETER

(71) Applicant: MASIMO CORPORATION, Irvine, CA (US)

(72) Inventors: Thomas B. Blank, Laguna Beach, CA (US); Gregory A. Olsen, Lake Forest, CA (US); Cristiano Dalvi, Lake Forest, CA (US); Hung T. Vo, Fountain Valley, CA (US)

(21) Appl. No.: 18/655,099

(22) Filed: May 3, 2024

Related U.S. Application Data
(63) Continuation of application No. 17/229,217, filed on Apr. 13, 2021, now Pat. No. 12,011,292.
Application No. 17/229,217 is a continuation of application No. 16/261,394, filed on Jan. 29, 2019, now Pat. No. 11,000,232.
Application No. 16/261,394 is a division of application No. 14/743,479, filed on Jun. 18, 2015, now Pat. No. 10,231,670.
(60) Provisional application No. 62/014,611, filed on Jun. 19, 2014.

(51) Int. Cl. A61B 5/00; A61B 5/1455

(52) U.S. Cl. CPC A61B 5/6844; A61B 5/14552; A61B 5/6826; A61B 5/684; A61B 2562/0257

(57) ABSTRACT

Systems and methods are disclosed for proximity sensing in physiological sensors, and more specifically to using one or more proximity sensors located on or within a physiological sensor to determine the positioning of the physiological sensor on a patient measurement site. Accurate placement of a physiological sensor on the patient measurement site is a key factor in obtaining reliable measurement of physiological parameters of the patient. Proper alignment between a measurement site and a sensor optical assembly provides more accurate physiological measurement data. This alignment can be determined based on data from a proximity sensor or sensors placed on or within the physiological sensor.

### CROSS-REFERENCE TO RELATED APPLICATIONS

This application is a continuation of U.S. patent application Ser. No. 17/229,217, filed Apr. 13, 2021, titled “PROXIMITY SENSOR IN PULSE OXIMETER,” which is a continuation of U.S. patent application Ser. No. 16/261,394, filed Jan. 29, 2019, now U.S. Pat. No. 11,000,232, titled “PROXIMITY SENSOR IN PULSE OXIMETER,” which is a division of Ser. No. 14/743,479, filed Jun. 18, 2015, now U.S. Pat. No. 10,231,670, titled “PROXIMITY SENSOR IN PULSE OXIMETER,” which claims the benefit of U.S. Provisional Application Ser. No. 62/014,611, filed Jun. 19, 2014, titled “PROXIMITY SENSOR IN PULSE OXIMETER;” the entire disclosures of which are hereby incorporated by reference.

### TECHNICAL FIELD

The systems and methods disclosed herein are directed to patient monitoring, and, more particularly, to pulse oximeter patient monitors capable of capacitive proximity detection.

### BACKGROUND

The standard of care in caregiver environments includes patient monitoring through spectroscopic analysis using, for example, a pulse oximeter. Devices capable of spectroscopic analysis generally include a light source(s) transmitting optical radiation into or reflecting off a measurement site, such as, body tissue carrying pulsing blood. After attenuation by tissue and fluids of the measurement site, a photodetection device(s) detects the attenuated light and outputs a detector signal(s) responsive to the detected attenuated light. A signal processing device(s) process the detector(s) signal(s) and outputs a measurement indicative of a blood constituent of interest, such as glucose, oxygen, methemoglobin, total hemoglobin, other physiological parameters, or other data or combinations of data useful in determining a state or trend of wellness of a patient.

In noninvasive devices and methods, a sensor is often adapted to position a finger proximate the light source and light detector. For example, noninvasive finger clip sensors often include a clothespin-shaped housing that includes a contoured bed conforming generally to the shape of a finger.

Accurate determination of physiological measurements is often dependent upon proper application of the optical sensor to the measurement site. Clip-type pulse oximeter sensors typically include a physical stop near the hinge of the housing to indicate desired placement of a user's finger or other measurement site within the sensor. However, the physical stop does not ensure that the patient's finger is positioned far enough into the sensor. In addition, even if a sensor is initially placed correctly, movement, either of the patient or of the sensor during artificial pulsing, can longitudinally displace the patient's finger within the sensor. This can result in the light source and detector of the oximeter being positioned around a portion of the finger that provides inaccurate physiological measurements.

### SUMMARY

The foregoing and other problems are addressed, in some embodiments, by providing an oximeter with proximity sensing technology that can be used to determine whether the oximeter is correctly applied to a patient measurement site. For example, one or more capacitive sensor electrodes can provide an indication to a processor of the oximeter regarding whether the sensor is correctly positioned by sensing proximity to the skin of the measurement site. Capacitive sensor electrodes can provide data representing a distance or relative distance between the electrodes and skin of the measurement site. Due to the inverse relationship between capacitance and distance, the sensitivity to the distance between the measurement site and the capacitive sensor electrodes increases as the distance between the measurement site and the capacitive sensor electrodes decreases. Accordingly, in one embodiment, a plurality of capacitive sensor electrodes can be positioned at various locations within the oximeter housing to provide proximity accurate feedback when the measurement site is located at a number of different positions relative to the oximeter. Though discussed primarily herein in the context of capacitive sensor electrodes, proximity feedback in pulse oximeters can be provided in other examples by optical, mechanical, or electrical sensors interfacing with skin of a measurement site, or a combination of one or more of capacitive, optical, mechanical, and electrical sensors.

In some embodiments, capacitive sensor electrodes can be used to determine the longitudinal displacement of a patient's finger within a clip-type pulse oximeter sensor housing. Using the determined displacement, the oximeter can determine whether to provide an indication to the patient or physician to reposition the oximeter. The oximeter can additionally or alternatively use the determined displacement to determine whether to mechanically reposition the optical assembly of the oximeter relative to the patient's finger. In oximeters implementing artificial pulsing, the capacitive sensor electrodes can periodically or continuously monitor the longitudinal displacement of the patient's finger within the sensor housing to determine a probe off condition. These examples illustrate some of the many benefits of an oximeter sensor having proximity sensing technology for determining positioning of the sensor relative to a measurement site.

For purposes of summarizing the disclosure, certain aspects, advantages and novel features of the inventions have been described herein. It is to be understood that not necessarily all such advantages can be achieved in accordance with any particular embodiment of the inventions disclosed herein. Thus, the inventions disclosed herein can be embodied or carried out in a manner that achieves or optimizes one advantage or group of advantages as taught herein without necessarily achieving other advantages as can be taught or suggested herein.

### BRIEF DESCRIPTION OF THE DRAWINGS

Throughout the drawings, reference numbers can be re-used to indicate correspondence between referenced elements. The drawings are provided to illustrate embodiments of the inventions described herein and not to limit the scope thereof.

FIG. 1A illustrates a high-level diagram of an embodiment of a physiological sensor having proximity sensing capabilities positioned around a patient measurement site.

FIG. 1B illustrates another embodiment of the physiological sensor of FIG. 1A.

FIG. 2A illustrates a perspective view of an embodiment of a physiological sensor having proximity sensing capabilities.

FIGS. 2B and 2C illustrate an exploded view of two components of the physiological sensor of FIG. 2A when disassembled.

FIG. 3A illustrates a perspective view of another embodiment of a physiological sensor having proximity sensing capabilities.

FIG. 3B illustrates a perspective view of the physiological sensor of FIG. 3A in an open position.

FIG. 4 illustrates a high-level schematic block diagram of an embodiment of a physiological sensor having proximity sensing capabilities.

FIG. 5 illustrates a schematic diagram of an embodiment of a circuit for measuring proximity of a measurement site to a sensor.

FIG. 6 illustrates an example process for determining positioning during sensor application.

FIG. 7 illustrates an example process for repositioning an applied sensor based on proximity sensing.

FIG. 8 illustrates an example process for determining a probe off condition based on proximity sensing.

### DETAILED DESCRIPTION

### I. Introduction

Implementations described herein relate generally to proximity sensing in physiological sensors, and more specifically to using one or more proximity sensors located on or within a physiological sensor to determine the positioning of the physiological sensor on a patient measurement site. Accurate placement of a physiological sensor on the patient measurement site is a key factor in obtaining reliable measurement of physiological parameters of the patient. For example, in clip-type pulse oximeter sensors, longitudinal positioning of the patient's finger within the sensor housing determines which portion of the patient's finger is aligned with an optical assembly used to generate physiological measurement data for determining one or more physiological parameters. Proper alignment with the optical assembly covers a detector of the optical assembly with the patient's fingertip and reduces introduction of ambient light to the detector, and accordingly provides more accurate physiological measurement data. This alignment can be determined based on data from a proximity sensor or sensors placed on or within the physiological sensor. Suitable proximity sensors include one or more capacitive sensors, optical scanning sensors, electrical sensors, or mechanical contact sensors.

The proximity data generated by the proximity sensors on or within a pulse oximeter can enable provision of more accurate physiological measurement data. For example, the proximity sensors described herein can be used to provide an indication to the oximeter to generate feedback for a patient or physician to reposition an improperly aligned oximeter sensor. In another example, the proximity sensors can be used to provide alignment information for mechanically repositioning the optical assembly with respect to the oximeter housing and patient finger. As a further example, the proximity sensors can be used to determine a probe off condition indicating that physiological measurement data obtained during persistence of the probe off condition should be discarded due to improper alignment. In additional examples, the proximity sensors can be used to associate distance data with measurement data output by the sensor, e.g. for assigning a confidence value to the measurement data.

### II. Overview of Example Proximity Sensing Physiological Monitoring Systems

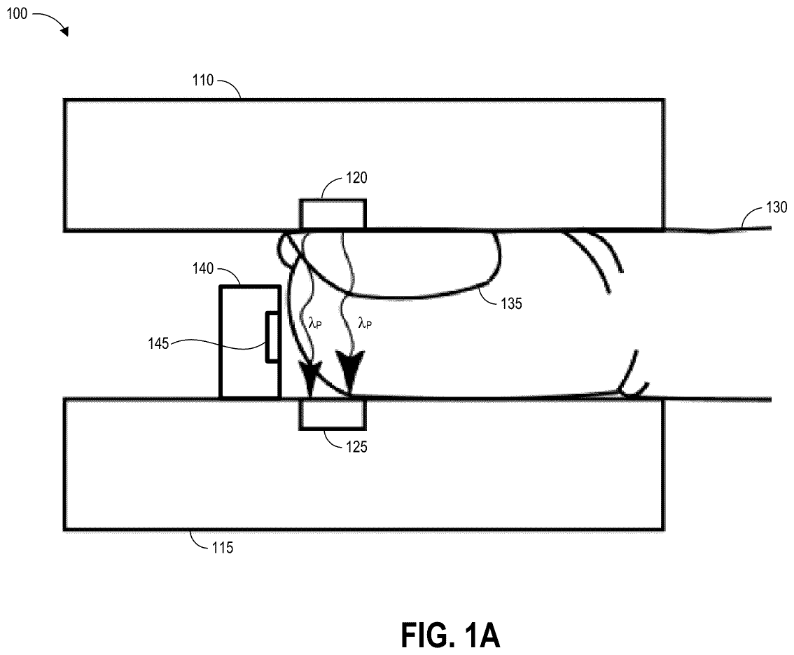

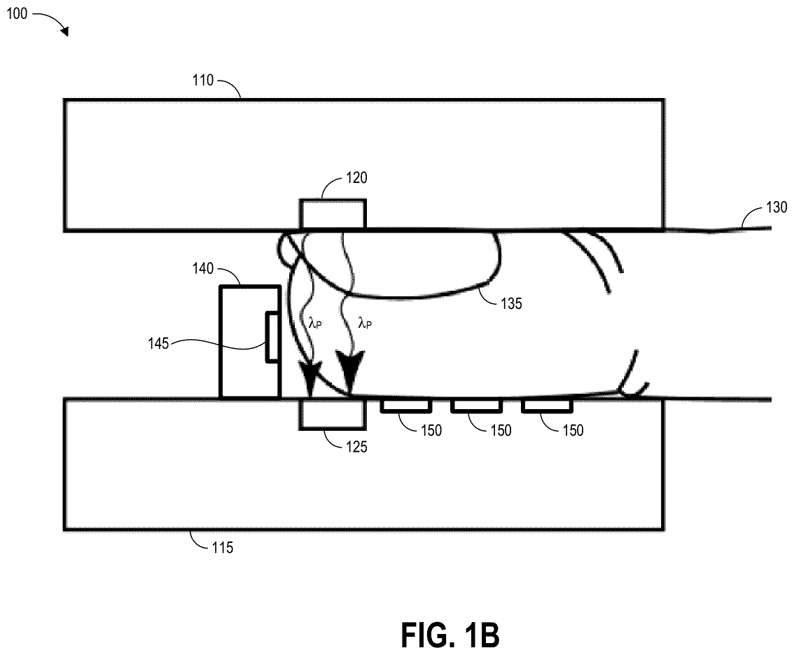

FIGS. 1A and 1B illustrate embodiments of a high-level diagram of an embodiment of a physiological sensor having proximity sensing capabilities positioned around a patient measurement site. The sensor 100 can include a first housing component 110 including an emitter 120 and a second housing component 115 including a detector 125. The sensor 100 can also include a physical stop 140 to guide the positioning of a patient finger 130 or other measurement site within the sensor 100. One or more proximity sensors 145 can be positioned on or within the sensor 100.

The emitter 120 can be configured to emit light having multiple primary wavelengths λ_{P} into the tissue of the patient finger 130 or other measurement site. The emitter 120 can be comprised of one or more devices such as semi-conductive light emitting diodes (LEDs), although it will be appreciated that other light generating devices may be used. The light emitter 120 may be chosen to emit light at a single known discrete wavelength, at multiple discrete wavelengths, or across a portion of the spectrum (such as that emitted by a “white light” LED), depending on the needs of the particular application. In one embodiment, the emitter 120 consists of two or more diodes emitting light energy in the infrared and red regions of the electromagnetic spectrum, and a parallel resistor (or resistors) used for security. The construction and operation of such light source drive circuitry is described in U.S. Pat. No. 5,758,644 incorporated herein by reference.

The detector 125 can be any suitable light energy detector responsive to light energy from the emitter, for example a semi-conductive photodetector. The emitter 120 and detector 125 can be aligned such that the detector 125 detects the emitted light after attenuation by the tissue of the patient finger 130.

The physical stop 140 can provide tactile feedback to a clinician or patient positioning the finger 130 within the sensor in order to achieve proper positioning. Proper positioning of the patient finger 130 or other tissue site relative to the detector 125 enables accurate physiological measurements to be made. In particular, the emitter 120 is placed so as to illuminate a blood-perfused tissue site 135, such as a nail bed, and the detector 125 is positioned so that only light transmitted by the emitter 120 and attenuated by pulsatile blood flowing within the tissue site 135 is received by the detector 125.

Physical stop 140 can prevent the tissue site 135 from being positioned beyond the emitter 120 and detector 125, that is, too far into the sensor 100. However, the physical stop 140 does not ensure that the tissue site 135 will be placed far enough within the sensor 100 for adequate transmission of light from the emitter 120 through the tissue site 135 to the detector 125 to enable clinically accurate physiological measurements. Accordingly, the sensor 100 can be provided with one or more proximity sensors 145 positioned within the sensor housing. The proximity sensor(s) 145 can be used to determine whether the tissue site 135 is positioned properly relative to the emitter 120 and detector 125.

Proximity sensor 145 can be a capacitive sensor, in some embodiments, that uses capacitance of the human body as an input to determine a distance between the patient's finger 130 and the proximity sensor 145. In another embodiment, proximity sensor 145 can be an optical scanning sensor, for example a camera or a near-infrared proximity sensor that uses light to determine how close or far the patient's finger 130 is from the proximity sensor 145. In still further embodiments, proximity sensor 145 can be a mechanical contact sensor that determines whether physical contact is made between the patient's finger 130 and the proximity sensor 145 mounted on the physical stop 140. Other sensors suitable for determining contact or distance between patient finger 130 and sensor 145 can be used in other embodiments.

As illustrated by FIG. 1A, the physical stop 140 can include a proximity sensor 145. The proximity sensor 145 of FIG. 1A can be used to determine the distance (or whether there is contact) between the location of the proximity sensor 145 on the physical stop 140 and the end of the patient's finger 130 nearest the stop 140. The proximity sensor 145 can be positioned on the physical stop 140 so as to sense or contact a portion of the patient fingertip below the nail, in particular for embodiments in which a capacitive proximity sensor is implemented. As illustrated by FIG. 1B, an additional array of proximity sensors 145 can be positioned longitudinally along the finger bed of the second housing component 115. The array 150 of proximity sensors can provide additional feedback regarding the longitudinal positioning of the patient finger 130 within the sensor 100.

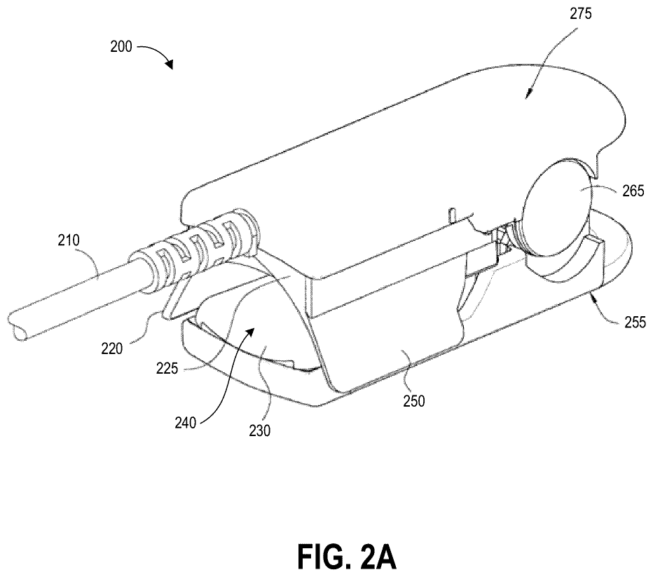

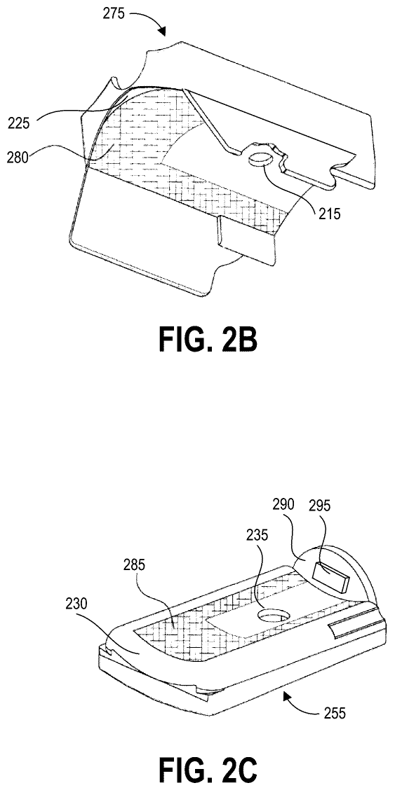

As illustrated in FIGS. 2A and 2B, an embodiment of a sensor 200 can include a two-piece housing and an electrical supply and signal cable 210. The housing consists of a first (upper) housing element 275 and a second (lower) housing element 255, which can be rotatably attached to one another via a pivot element 265. A light emitter can be disposed within the upper housing element 275, while a detector can be disposed within the lower housing element 255. The housing is adapted to receive the distal end of a finger as shown in the block diagrams of FIGS. 1A and 1B, with the “upper” housing element 275 engaging the upper surface of the finger, and the “lower” housing element engaging the lower surface of the finger. It will be recognized, however, that the sensor 200 may be used in any orientation, such as with the first housing element 275 being located below the second housing element 255. Furthermore, the light emitter may alternatively be placed in the lower housing element 255, and the detector in the upper housing element 275 if desired, subject to modification of other probe components as described further below. It is also noted that while the following discussion describes a series of exemplary embodiments based on measuring the optical characteristics of a finger, the sensor 200 may be adapted for use with any number of other body parts, such as earlobes or loose skin, with equal success. Additional details of an embodiment of the sensor are disclosed in U.S. Pat. No. 6,580,086 entitled “Shield Optical Probe and Method,” filed on Oct. 19, 1999 and assigned to Masimo Corporation, the entirety of which is hereby incorporated by reference.

The first and second housing elements 275, 255 can be generally rectangular in form with a pivot element 265 disposed near a common end of each of the elongate housing elements 275, 255. The two housing elements 275, 255 can be biased around the rotational axis of the pivot element by a biasing element, for example a hinge spring. The upper housing element 275 can be accordingly biased against the lower housing element 255 for secure placement on a patient finger or other measurement site. The user can grasp the sensor 200 between his or her fingers and separate the probe housing elements 275, 255 by applying force counter to the spring biasing force. In this fashion, the user simply grasps the sensor 200, opens it by applying a light force with the grasping fingers, and inserts the distal end of the patient's finger into the end 240 of the sensor 200. Once the finger is inserted into the sensor 200, the disproportionate compression of the finger (due to interaction of the angled housing elements 275, 255 and the substantially cylindrical finger) and the aforementioned bias spring separating force act to lower housing elements 275, 255 substantially parallel to each other, allowing more of the surface area of the upper and lower support surface elements 225, 230 to contact the finger, and for more even pressure distribution thereon. This assists with accurate positioning of the finger with respect to the emitter and detector for clinically accurate physiological measurement readings using the sensor 200.

As shown in more detail in FIGS. 2B and 2C, the housing elements 275, 255 can include first (upper) and second (lower) support surface elements 225, 230, respectively, which provide support and alignment for the tissue material, such as the finger, when the sensor 200 is clamped thereon. The upper support surface element 225 can be fashioned from a substantially pliable polymer such as silicone rubber, so as to permit some deformation of the element 225 when in contact with the fairly rigid upper portion 280 of the patient's finger. The upper support surface element 225 further includes an optical energy shield 250 which protrudes from the upper support surface element 225. The shield 250 can be sized and shaped so as to conform substantially to the outer circumference of the patient's finger, providing at least a partial seal against ambient light incident on the probe exterior and otherwise exposed portions of the finger. In this fashion, patients having fingers of different circumferences can be accommodated with the same sensor 200 due to use of shield 250. The lower surface element 230 can be fashioned from a substantially solid and rigid (i.e., higher durometer) polymer. This harder, solid polymer can be used for the lower surface element 230 since the lower portion of the finger is generally more fleshy and deformable, thereby allowing the skin and tissue material thereof to deform and contour to the shape of the inner region 285 of the lower surface element. The inner regions 280, 285 can be contoured to assist in mitigating the effects of patient movement during operation of the sensor 200. Accordingly, the construction of the sensor 200 provides some alignment between the patient finger and the sensor 200 for proper positioning relative to the emitter and detector.

The upper surface element 225 includes an aperture 215 for transmission of light therethrough after emission by the emitter in the upper housing element 275. The lower surface element 230 includes an aperture 235 aligned with the detector for transmission of light therethrough after passing through the measurement site. The apertures 215, 235 allow for light energy to be transmitted between the light emitter and tissue material of the measurement site, and similarly between the tissue material and detector. The first aperture 215 is also axially located with the second aperture 235 in the vertical dimension, such that when the probe 100 is in the closed configuration with the patient's finger disposed between the upper and lower surface support elements 225, 230, light emitted by the light source through the first aperture 215 is transmitted through the finger and the second aperture 235 and received by the detector. Hence, the light source, first aperture 215, second aperture 235, and detector are substantially axial in this configuration.

The lower support element 230 is further provided with a physical stop 290 disposed near the pivot element of the sensor 200. The physical stop 290 is oriented vertically with respect to the lower support element 230 so as to stop the distal end of the patient's finger from being inserted into the probe past a certain point, thereby facilitating proper alignment of the finger within the sensor 200, especially with respect to the source and detector apertures 215, 235. While the present embodiment uses a semi-circular tab as the physical stop 290, it will be recognized that other configurations and locations of the physical stop 290 may be used. For example, the tab could be bifurcated with a portion being located on the upper support surface element 230, and a portion on the lower support surface element 225. Alternatively, the positioning element could be in the form of a tapered collar which receives, aligns, and restrains only the distal portion of the patient's finger. Many such alternative embodiments of the positioning element are possible, and considered to be within the scope of the present invention.

A proximity sensor 295 is positioned on the physical stop 290 that can be used to determine whether the patient finger or other measurement site is positioned properly within the sensor 200. As discussed above, proximity sensor 295 can be a capacitive sensor, an optical scanning sensor, or a mechanical contact sensor, or a combination of two or more of these sensors in some embodiments. Proximity sensor 295 provides feedback regarding distance or contact between the patient finger and the proximity sensor 295 located on physical stop 290, and accordingly can be used to determine whether the patient finger is aligned with the emitter and detector of the sensor 200. This feedback can be used to provide a repositioning indication to the user of the sensor 200, to mechanically reposition the optical components of the sensor 200, or for data filtering, as described in more detail below. Accordingly, the feedback from the proximity sensor 295 can enable more accurate physiological measurements using the sensor 200.

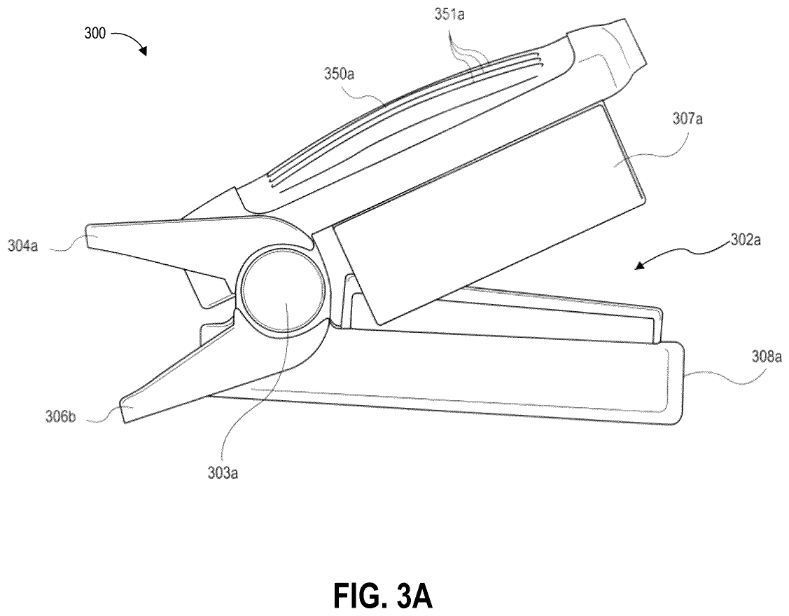

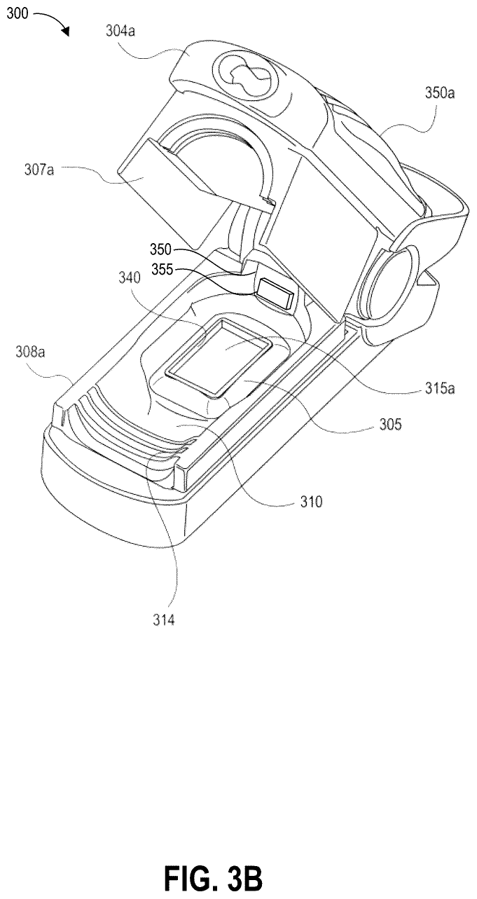

FIGS. 3A and 3B illustrate another embodiment of a sensor 300. The sensor 300 shown can include all of the features of the sensors 100 and 200 described above.

Referring to FIG. 3A, the sensor 300 in the depicted embodiment is a clothespin-shaped clip sensor that includes an enclosure 302a for receiving a patient's finger. The enclosure 302a is formed by an upper housing or emitter shell 304a, which is pivotably connected with a lower housing or detector shell 306a. The emitter shell 304a can be biased with the detector shell 306a to close together around a pivot point 303a and thereby sandwich finger tissue between the emitter and detector shells 304a, 306a.

In an embodiment, the pivot point 303a advantageously includes a pivot capable of adjusting the relationship between the emitter and detector housings 304a, 306a to effectively level the sections when applied to a tissue site. In another embodiment, the sensor 300 includes some or all features of the finger clip described in U.S. Pat. No. 8,437,825, entitled “Contoured Protrusion for Improving Spectroscopic Measurement of Blood Constituents,” filed on Jul. 2, 2009 and assigned to Cercacor Laboratories, the entirety of which is hereby incorporated by reference. For example, the sensor 300 can include a spring that causes finger clip forces to be distributed along the finger.

The emitter housing 304a can position and house various emitter components of the sensor 301a. It can be constructed of reflective material (e.g., white silicone or plastic) and/or can be metallic or include metallized plastic (e.g., including carbon and aluminum) to possibly serve as a heat sink including one or more fins 351a. The emitter housing 304a can also include absorbing opaque material, such as, for example, black or grey colored material, at various areas, such as on one or more flaps 307a, to reduce ambient light entering the sensor 301a. The emitter housing 304a can also include optical shield 307a to block ambient light from entering the sensor 300.

The detector housing 306a can position and house one or more detector portions of the sensor 301a. The detector housing 306a can be constructed of reflective material, such as white silicone or plastic. As noted, such materials can increase the usable signal at a detector by forcing light back into the tissue and measurement site (see FIG. 1). The detector housing 306a can also include absorbing opaque material at various areas, such as lower area 308a, to reduce ambient light entering the sensor 301a.

Referring to FIG. 3B, an example of finger bed 310 is shown in the sensor 301b. The finger bed 310 includes a generally curved surface shaped generally to receive tissue, such as a human digit. The finger bed 310 includes one or more ridges or channels 314. Each of the ridges 314 has a generally convex shape that can facilitate increasing traction or gripping of the patient's finger to the finger bed. Advantageously, the ridges 314 can improve the accuracy of spectroscopic analysis in certain embodiments by reducing noise that can result from a measurement site moving or shaking loose inside of the sensor 301a. The ridges 314 can be made from reflective or opaque materials in some embodiments to further increase signal to noise ration (SNR). In other implementations, other surface shapes can be used, such as, for example, generally flat, concave, or convex finger beds 310.

Finger bed 310 can also include an embodiment of a tissue thickness adjuster or protrusion 305. The protrusion 305 includes a measurement site contact area 370 that can contact body tissue of a measurement site. The protrusion 305 can be removed from or integrated with the finger bed 310. Interchangeable, different shaped protrusions 305 can also be provided, which can correspond to different finger shapes, characteristics, opacity, sizes, or the like. In some embodiments, protrusion 305 can include one or more proximity sensors to determine positioning of a patient finger over the protrusion 305.

The detector housing 306a can also include a physical stop 350 to prevent a patient's finger from being longitudinally placed too far into the sensor for proper alignment with the emitter and detector. Proper positioning of the patient finger or other tissue site relative to the detector enables accurate physiological measurements to be made. The physical stop 350 can include one or more proximity sensors 355 for determining distance or contact between the end of the patient's finger and the proximity sensor 355 to provide feedback regarding alignment of the patient's finger with the emitter and detector. As described above, the proximity sensor 355 can be a capacitive sensor, optical scanning sensor, or mechanical contact sensor. Data from proximity sensor 355 can be used to determine whether the patient finger is aligned with the emitter and detector of the sensor 300. This feedback can be used to provide a repositioning indication to the user of the sensor 300, to mechanically reposition the optical components of the sensor 200, or for data filtering, as described in more detail below. Accordingly, the feedback from the proximity sensor 295 can enable more accurate physiological measurements using the sensor 200.

In a vascular bed the arterial vasculature is coupled mechanically to the venous vasculature through the tissues. Although this coupling is small, the optical arterial pulse, e.g. photo-plethysmograph, has invariably a small venous component. This component is not fixed across subjects but its average is indirectly calibrated for in the saturation calibration curve. Its effect on the arterial pulse is proportional to the coupling size as well as the difference between the arterial and venous saturations at the site. Its effects may be explained by the reduction in the optical effect of venous coupling as the delta saturation between the arterial and the venous is reduced due to the increase in availability of plasma oxygen. Under this condition, the venous blood will look, optically, a lot like the arterial blood. Hence, the size of the Red photo-plethysmograph signal will shrink with respect to the IR indicating a shrinking ΔSat, i.e. higher venous saturation. In 1995, Masimo Corporation (Masimo) introduced a new technique for calculation the venous oxygen saturation (SpvO₂) by introducing an artificial pulse into the digit (see, e.g., U.S. Pat. No. 5,638,816, incorporated herein by reference).

The sensor 300 depicted in FIGS. 3A and 3B can be capable of introducing an artificial pulse into the measurement site. The sensor 300 can induce an artificial pulse at a frequency distinguishable from the frequency of a human arterial pulse. As a result, information related to both the arterial pulse as well as the artificial pulse is recoverable from the body. The redundant nature of both pieces of information provide additional information useful in determining physiological parameters.

Introducing an artificial excitation can cause perturbations in the blood flow similar to the effects of a heart beat. These artificial excitations can be used as an alternative to the natural pulse rate or in addition to the natural pulse rate. Artificial excitations have the added benefit that the excitations introduced are introduced at known frequencies. Thus, it is not necessary to first determine the pulse rate of an individual.

However, the movement required to generate the artificial pulse can cause movement of the sensor relative to the patient, introducing the variable of distance into denoising equations for the resultant sensor data. For example, the artificial pulse can dislodge the sensor 300 from the patient measurement site or misalign the measurement site with the emitter and detector of the sensor 300. Accordingly, sensor data from active pulse sensors typically is filtered to determine probe off and probe on conditions, where probe on conditions indicate reliable sensor data and probe off conditions include their ordinary broad meaning known to one of skill in the art, including designating improper application of an optical probe to a measurement site, for example due to movement of the sensor relative to the patient caused by artificial pulsing.

Data from the proximity sensor 355 within the sensor 300 can be used instead of or in addition to more complex data processing methods to determine a probe off condition due to improper alignment of the measurement site and the sensor. For example, if data from the proximity sensor 355 indicates that the measurement site is not properly aligned with the sensor 300, then the sensor 300 can determine a probe off condition for the duration of such data from the proximity sensor 335. Accordingly, data from proximity sensor 355 can provide a computationally simple and accurate means for determining the probe off condition.

In some embodiments, instead of or in addition to using proximity data to determine a probe off condition, data from the proximity sensor 355 can be used to generate a confidence value indicating potential accuracy of measurement data, e.g., plethysmographic data output by a pulse oximeter. For example, during operation a sensor can supply associated proximity data and plethysmographic data to a patient monitor without utilizing probe-off detection or probe repositioning. The patient monitor can then determine a confidence value for each portion of the plethysmographic data based on the associated proximity data, for example a measured distance between a fingertip and a capacitive proximity sensor measured at substantially the same time as when the plethysmographic data was captured. Using the confidence values for the plethysmographic data (e.g., to discard certain portions of the plethysmographic data or to assign different weights to different portions of the plethysmographic data), the patient monitor can estimate pulse rates, respiration rates, and the like for the patient.

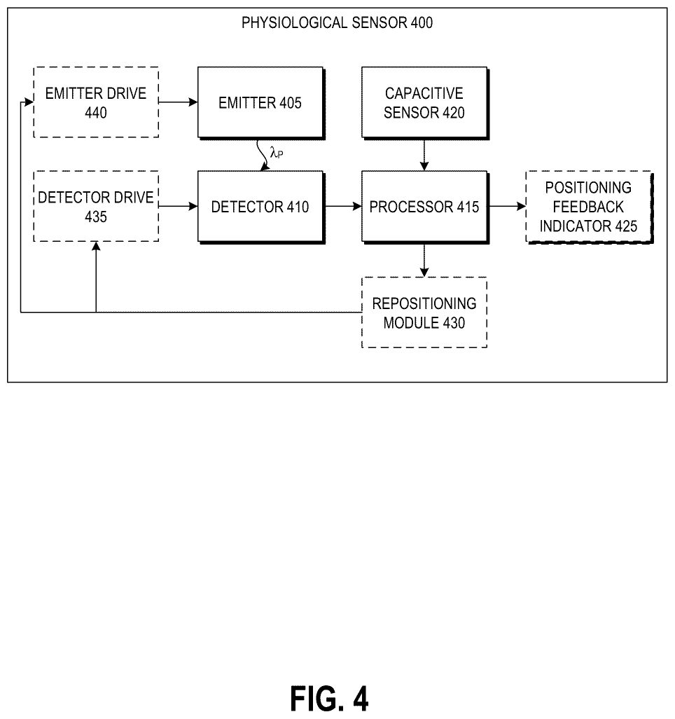

FIG. 4 illustrates a high-level schematic block diagram of an embodiment of a physiological sensor 400 having proximity sensing capabilities. The physiological sensor 400 includes an emitter 405, detector 410, processor 415, and capacitive sensor 420. In other embodiments the capacitive sensor 420 can be replaced or supplemented by the other types of proximity sensors discussed herein. Various embodiments of the sensor 400 can also include one or more of a set of optional components such as positioning feedback indicator 425, repositioning module 430, emitter drive 440, or detector drive 435. Though not illustrated, the sensor 400 can also include memory, a display, connection ports, user interface elements, alarms, and other components.

The emitter 405 can be capable of emitting light at one or a plurality of wavelengths λ_{P} for noninvasive measurement of constituents of blood flowing within the tissue of a patient measurement site. The emitter 405 and detector 410 can be aligned so that some or all of the light λ_{P} is incident on detector 410 after passage through the measurement site and attenuation by the constituents of the blood.

Intensity signals representing the light λ_{P} incident on detector 410 can be sent to processor 415. Processor 415 can analyze the intensity signals and determine one or more physiological parameter values. For example, processor 415 can calculate a ratio of detected red and infrared intensities, and an arterial oxygen saturation value is empirically determined based on that ratio. Processor 415 can also perform noise filtering on the raw intensity signal data, and can determine probe off and probe on conditions for purposes of excluding unreliable data from use in calculating physiological parameters. Data from the capacitive sensor 420 can provide feedback regarding the distance of a patient measurement from the capacitive sensor, which can be compared to a threshold to determine whether the sensor and finger are improperly aligned. The determination of improper alignment can be used by the processor 415 in one embodiment to determine a probe off condition.

In some embodiments, the processor 415 can generate a repositioning signal based at least partly on the determination of improper alignment from capacitive sensor data. This signal can be sent, in one embodiment, to positioning feedback indicator 425. Positioning feedback indicator 425 can provide visual, audible, or tactile feedback to a user of sensor 400 to indicate whether the sensor should be repositioned. For example, sensor 400 may emit a beep or audible alarm when the sensor 400 is improperly positioned. Sensor 400 may also include one or more visual indications, for example LED lights, that can be used to provide positioning feedback to the user. For instance, a green light may be illuminated to indicate to the user that the sensor 400 is properly positioned. A red light may be illuminated to indicate to the user that sensor 400 is improperly positioned. As another example, sensor 400 may output text or voice commands indicating how far the user must adjust the sensor 400 in order to achieve proper alignment based on the proximity sensor data. Positioning feedback indicator 425 can provide an initial positioning indication when the user first applies the sensor in one embodiment. In some embodiments, positioning feedback indicator 425 can periodically or continuously output positioning feedback to the user, for example for active pulse sensors introducing an artificial pulse during measurement.

The repositioning signal can be sent, in another embodiment, to repositioning module 430. Repositioning module 430 can use the repositioning signal to generate instructions for mechanical repositioning of the emitter 405 and detector 410, for example using emitter drive 440 and detector drive 435. In some embodiments, emitter drive 440 and detector drive 435 could be implemented together as a single optical assembly drive. Emitter drive 440 and detector drive 435 can be used to reposition the emitter 405 and detector 410, respectively, according to the data provided by the capacitive sensor 420 in order to align the emitter 405 and detector 410 with the measurement site.

As discussed above with respect to the sensors of FIGS. 1A-3B, the capacitive sensor 420 can be placed within the body of the sensor 400 to gauge longitudinal displacement of a patient's finger relative to the sensor 400 and, more specifically, the optical assembly including the emitter 405 and detector 410. In some embodiments, multiple capacitive sensors can be positioned along a longitudinal axis of the sensor 400 for additional data as the patient's finger moves within the sensor housing.

### III. Overview of Example Capacitive Proximity Sensor

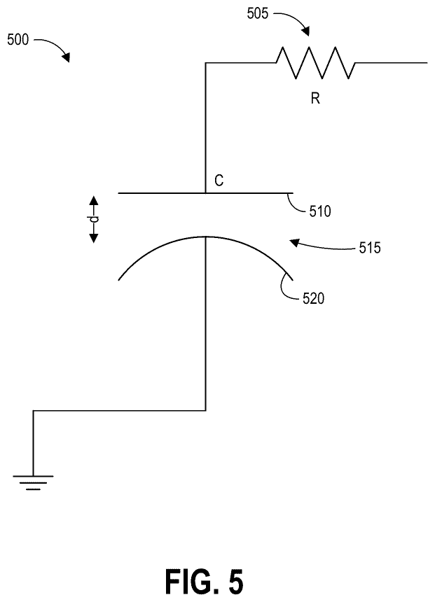

FIG. 5 illustrates a schematic diagram of an embodiment of a circuit 500 that can be used to determine proximity of a patient measurement site to a physiological sensor. The circuit includes a capacitor 515 and a resistor 505. Here the capacitance, C, can be calculated according to Equation (1), below:

$$C ∝ \frac{A}{d} \qquad ( 1 )$$

where the capacitance, C, is proportional to the area, A, of the capacitor divided by the distance, d, between the capacitor plates. The capacitor 515 has two plates 510, 520. The first capacitive plate 510 is integrated or connected to the physiological sensor, for example a physical stop near the hinge of the housing of clip-type pulse oximeter, or in an array along the longitudinal axis of the surface of a finger bed of clip-type pulse oximeter. The second capacitor plate 520 is the skin surface of the patient measurement site, with the assumption that the patient is a capacitor. A typical adult has a capacitance of about 120 pF, however capacitance of different people will vary. Accordingly, the distance, d, between the capacitor plates 510, 520 represents the distance between the location of the capacitive sensor within the physiological sensor and the surface of the patient measurement site. In order to obtain the value of the distance, d, from Equation (1), the values of both the capacitance, C, and the area, A, must be known. The area can be predetermined based on the dimensions of the first capacitive plate 510.

The capacitance can be determined from the value of the resistance, R, of the resistor 505 and the value of a time constant, τ, of the circuit 500. The time constant of the circuit 500 can be calculated according to Equation (2), below:

$$τ = RC \qquad ( 2 )$$

where the time constant, τ, is equal to the resistance, R, times the capacitance, C. Hence, the distance, d, between the capacitor plates 510, 520 can be calculated through the combination of Equations (1) and (2) through the measurement of the circuit time constant. Accordingly, as a distance between a patient measurement site, such as a digit of a hand, and the capacitor plate 510 decreases, the time constant τ increases and the capacitance C increases.

In one embodiment, the time constant τ can be determined from the time required to trip a set voltage level, such as about 2.2 volts, given a power supply of known power, such as about 3.3 volts. Although not illustrated, a processor or microprocessor can be in communication with the circuit 500 to measure the time constant. The determined time constant τ can be used to calculate the capacitance, C, using Equation (2). The capacitance C can then used to calculate the distance or relative distance through Equation (1). Accordingly, as a distance between a patient measurement site, such as a digit of a hand, and the capacitor plate 510 decreases, the time constant τ increases and the capacitance C increases.

The value of the distance d can be used to provide a repositioning indication to the patient or physician, to mechanically reposition the optical assembly of an oximeter sensor, or to determine a probe off condition for removing potentially unreliable data from a physiological measurement data set. In some embodiments, a number of circuits 500 can be provided within an oximeter for precise determination of longitudinal displacement of a patient finger within the oximeter housing. Accordingly, at least one of the repositioning indication, optical assembly repositioning, or probe off can be based on a number of distance values corresponding to each of the circuits. Although an example capacitive circuit 500 is depicted, this is for purposes of illustration and in other embodiments other capacitive circuit arrangements can be used. For example, other suitable circuits can include amplifiers, additional resistors, and other elements, for instance to amply the signal to noise and to minimize changes to capacitance based on environmental changes.

### IV. Overview of Example Proximity-Sensing Physiological Monitoring Processes

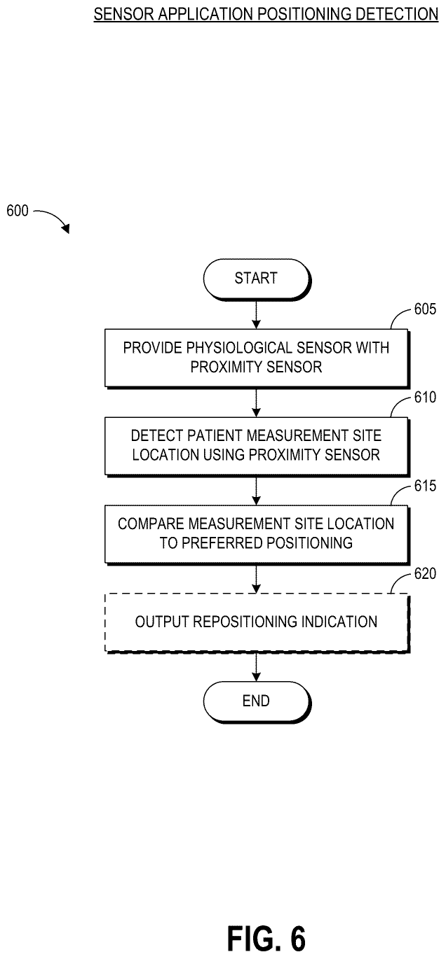

FIG. 6 illustrates an example process 600 for determining positioning during sensor application. The process 600 can be implemented on any physiological sensor including proximity sensing capabilities, for example the physiological sensors illustrated in FIGS. 1A-FIG. 4.

At block 605, a physiological sensor is provided with at least one proximity sensor. For example, one proximity sensor or an array of proximity sensors can be provided including capacitive, optical, electrical, or mechanical proximity sensors, or a combination thereof. The proximity sensor can be configured to determine distance or contact between a measurement site and itself, and can be positioned within the sensor so as to provide feedback regarding alignment of the measurement site and optical components of the sensor.

At block 610, the patient measurement site location is detected using the proximity sensor. For example, using a capacitive proximity sensor, a distance between a capacitive plate in the sensor and the capacitive skin of the patient can be determined. An optical scanning proximity sensor can also determine a distance between the sensor and a patient measurement site. In another example, using a mechanical contact proximity sensor, it can be determined whether the patient measurement site has contacted the mechanical contact proximity sensor, such as by depressing a button.

At block 615, the measurement site location can be compared to preferred positioning. The preferred positioning can include a range of placements relative to the optical components of the sensor that are likely to produce clinically accurate physiological measurements. To illustrate, if a capacitive proximity sensor is used on a physical stop in a pulse oximeter finger clip sensor such as is depicted in FIG. 2A, 2B, 3A, or 3B, a distance of approximately 4 mm-10 mm between the capacitive proximity sensor and the patient fingertip can correspond to proper positioning of an adult finger with respect to an LED emitter and detector. In one embodiment, a distance of approximately 6 mm between the capacitive sensor and the fingertip can indicate preferred positioning of the finger within the sensor. Other ranges can be used to indicate preferred placement in other sensor configurations, for other finger sizes, or for other measurement sites.

At block 620, the sensor can optionally cause the sensor to output a repositioning indication. For instance, in one embodiment of a pulse oximeter finger clip sensor such as is depicted in FIG. 2A, 2B, 3A, or 3B having a capacitive sensor on a physical stop, if the capacitive proximity sensor data indicates that the patient fingertip is closer than approximately 4 mm to the capacitive sensor or farther than approximately 10 mm from the capacitive sensor, the sensor may output a repositioning indication. In some embodiments, the sensor may output a first positioning indication when the sensor is correctly positioned and may output a second positioning indication when the sensor is incorrectly positioned. The second positioning indication may be a visual, auditory, or tactile signal alerting the user that the sensor is incorrectly positioned, and in one example may include specific instructions regarding how to reposition the sensor for proper placement.

Process 600 can be implemented when a user first applies a sensor to a measurement site to indicate proper positioning. In some embodiments, blocks 610-620 may be repeated one or more times during measurement to gauge whether the sensor positioning continues to be proper or whether the sensor should be repositioned. Repetition of this measurement site location detection and comparison portion of process 600 can be beneficial, in one example, in active pulse oximeters that introduce an artificial pulse to a measurement site and therefore possibly introduce movement of the sensor relative to the measurement site. Repetition of blocks 610-620 can also be beneficial, in another example, in patient monitoring situations in which the patient can move during the time in which the sensor is worn and therefore may dislodge the sensor.

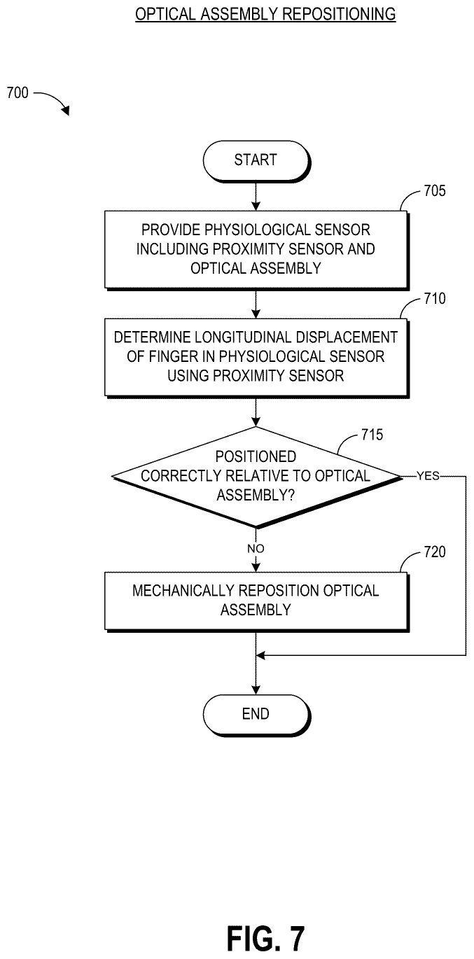

FIG. 7 illustrates an example process 700 for repositioning an applied sensor based on proximity sensing. The process 700 can be implemented by the components of any physiological sensor including proximity sensing capabilities, for example the physiological sensors illustrated in FIGS. 1A-FIG. 4.

At block 705, a physiological sensor is provided with at least one proximity sensor. For example, one proximity sensor or an array of proximity sensors can be provided including capacitive, optical, electrical, or mechanical proximity sensors, or a combination thereof. The proximity sensor can be configured to determine distance or contact between a measurement site and itself, and can be positioned within the sensor so as to provide feedback regarding alignment of the measurement site and optical components of the sensor. The physiological sensor also includes an optical assembly, for instance an aligned emitter and detector as discussed above with respect to Figures IA-FIG. 4, for generating intensity signals from which to determine physiological signals.

At block 710, the sensor determines longitudinal displacement of a patient finger in the physiological sensor using the proximity sensor. Many pulse oximeters are contoured to naturally center a patient finger within the sensor, and therefore the proximity sensing discussed herein focuses primarily on determining longitudinal displacement. However, in other sensor embodiments displacement of the measurement site along a vertical or horizontal axis of the sensor may alternatively or additionally be determined. For example, using a capacitive proximity sensor, a distance between a capacitive plate in the sensor and the capacitive skin of the patient can be determined. An optical scanning proximity sensor can also determine a distance between the sensor and a patient measurement site. In another example, using a mechanical contact proximity sensor, it can be determined whether the patient measurement site has contacted the mechanical contact proximity sensor, such as by depressing a button.

At block 715, the sensor determines whether it the patient measurement site is correctly positioned relative to the optical assembly based on the proximity sensor data. As discussed above, for capacitive proximity sensors the distance between the capacitive proximity sensor and the skin of the measurement site can be compared to a threshold or to a range of acceptable positions to determine whether the measurement site is correctly positioned. For a mechanical contact sensor producing a binary contact or no contact output, the correct positioning determination may be made based on the binary output.

If the measurement site is positioned correctly relative to the optical assembly, then the process 700 ends. If the measurement site is not positioned correctly relative to the optical assembly, then the process 700 transitions to block 720 in which the sensor mechanically repositions the optical assembly to achieve proper alignment with the measurement site. For example, capacitive sensing data can be used to determine a distance to mechanically move the optical assembly along the longitudinal axis of the sensor.

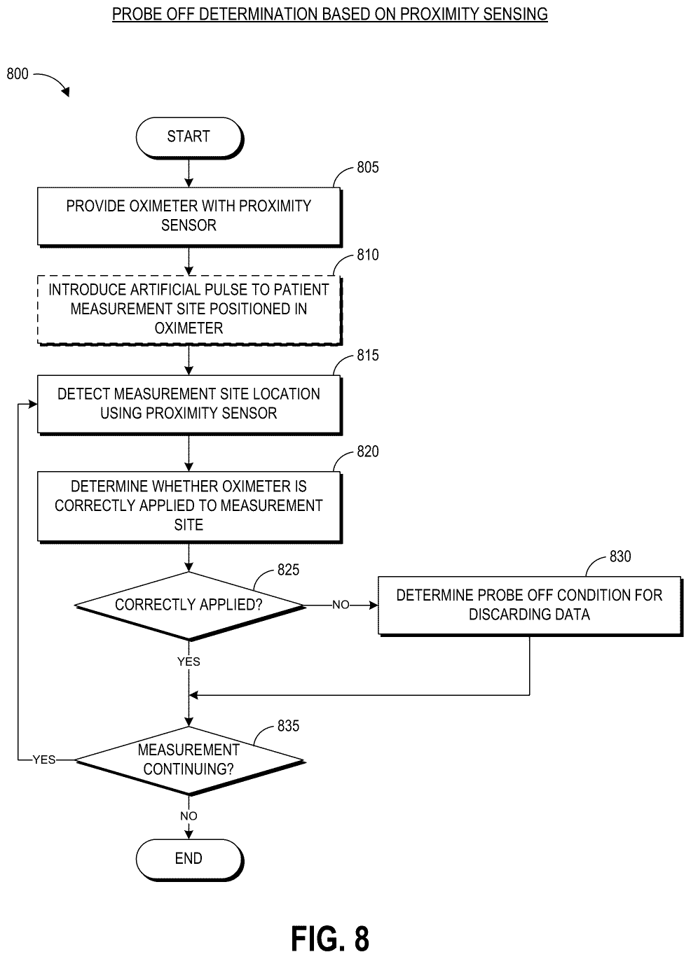

FIG. 8 illustrates an example process 800 for determining a probe off condition based on proximity sensing. The process 800 can be implemented by the components of any physiological sensor including proximity sensing capabilities, for example the physiological sensors illustrated in FIGS. 1A-FIG. 4. Some embodiments of process 800 can be implemented on an active pulse sensor capable of introducing an artificial pulse into a measurement site.

At block 805, a physiological sensor is provided with at least one proximity sensor. For example, one proximity sensor or an array of proximity sensors can be provided including capacitive, optical, electrical, or mechanical proximity sensors, or a combination thereof. The proximity sensor can be configured to determine distance or contact between a measurement site and itself, and can be positioned within the sensor so as to provide feedback regarding alignment of the measurement site and optical components of the sensor.

At block 810, the sensor optionally introduces an artificial pulse to the patient measurement site, such as a finger positioned within a pulse oximeter. As discussed above, the artificial pulse can assist in obtaining more reliable measurement data, however it can also cause movement of the sensor relative to the measurement site, thereby affecting proper positioning of the sensor on the measurement site.

At block 815, the patient measurement site location is detected using the proximity sensor. As discussed above, in some embodiments of a finger clip pulse oximeter the sensor may be placed on a physical stop, and may be oriented to determine longitudinal displacement of the patient finger within the sensor relative to the optical assembly. In one example, using a capacitive proximity sensor, a distance between a capacitive plate in the sensor and the capacitive skin of the patient can be determined. An optical scanning proximity sensor can also determine a distance between the sensor and a patient measurement site. In another example, using a mechanical contact proximity sensor, it can be determined whether the patient measurement site has contacted the mechanical contact proximity sensor, such as by depressing a button.

At block 820, the measurement site location can be compared to preferred positioning data to determine whether the oximeter is correctly applied to the measurement site. Preferred positioning data can be stored in memory of the oximeter, for example a read-only memory (ROM). The preferred positioning can include a range of placements relative to the optical components of the sensor that are likely to produce clinically accurate physiological measurements. In one embodiment, if a capacitive proximity sensor is used on a physical stop in a pulse oximeter finger clip sensor such as is depicted in FIG. 2A, 2B, 3A, or 3B, a distance of approximately 4 mm-10 mm between the capacitive proximity sensor and the patient fingertip can correspond to proper positioning of an adult finger with respect to an LED emitter and detector. As an example, a distance of approximately 6 mm between the capacitive sensor and the fingertip can indicate preferred positioning of the finger within the sensor. As discussed above, other ranges can be used to indicate preferred placement in other sensor configurations, for other finger sizes, or for other measurement sites.

At block 825, the sensor determines based on the comparison of block 810 whether the oximeter is correctly applied to the measurement site. If the sensor is not correctly applied, then the process 800 transitions to block 830 in which the sensor, for example a processor of the sensor or a processor of another device connected to and receiving data from the sensor, determines a probe off condition for discarding the data taken while the sensor was not correctly applied. In some embodiments, a default may be to determine a probe on condition for including data, and data may only be discarded when a probe off condition is determined for a time or range of times during which sensor data was gathered at improper positioning.

After determining the probe off condition at block 830, or if the sensor is determined at block 825 to be correctly applied, the process 800 transitions to block 835 to determine whether measurement is continuing. If measurement continues, then the process 800 loops back to block 815 to determine the measurement site location and to repeat blocks 820 and 825. Once measurement has concluded, for instance when the sensor is powered off or if the sensor detects that the patient measurement site is no longer at least partially within the sensor housing, then the process 800 ends.

### V. Terminology

Although many of the examples discussed herein are in the context of pulse oximetry, this is for illustrative purposes only. The sensors, signal conditioning techniques, and proximity sensing applications discussed herein can be adapted for other physiological sensors monitoring other physiological parameters or multiple physiological parameters.

Many other variations than those described herein will be apparent from this disclosure. For example, depending on the embodiment, certain acts, events, or functions of any of the algorithms described herein can be performed in a different sequence, can be added, merged, or left out all together (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. For example, any of the signal processing algorithms described herein can be implemented in analog circuitry. A computing environment can include any type of computer system, including, but not limited to, a computer system based on a microprocessor, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others. It should be emphasized that many variations and modifications may be made to the above-described embodiments, the elements of which are to be understood as being among other acceptable examples. All such modifications and variations are intended to be included herein within the scope of this disclosure and protected by the following claims.

1-20. (canceled)

21. A system for monitoring a physiological parameter, the system comprising:
one or more sensors configured to generate first sensor data and second sensor data, the first sensor data being indicative of a distance between the one or more sensors and a measurement site, the second sensor data being usable to determine one or more estimated values of a physiological parameter; and
one or more processors configured to:
determine, from the first sensor data, that a first set of the second sensor data is generated when the distance between the one or more sensors and the measurement site satisfies a threshold;
determine, from the first sensor data, that a second set of the second sensor data is generated when the distance between the one or more sensors and the measurement site does not satisfy the threshold;
determine, from the first set of the second sensor data and the second set of the second sensor data, an estimated value of the physiological parameter by weighting the first set of the second sensor data differently from the second set of the second sensor data; and
output the estimated value for presentation to a user.

22. The system of claim 21, wherein the one or more processors are configured to:
determine, from the first sensor data, that a third set of the second sensor data is generated when the first sensor data indicates the one or more sensors are positioned away from the measurement site; and
responsive to determining that the third set of the second sensor data is generated when the first sensor data indicates the one or more sensors are positioned away from the measurement site, refrain from using the third set of the second sensor data to determine any estimated values of the physiological parameter.

23. The system of claim 21, wherein the one or more processors are configured to:
determine, from the first sensor data, a first confidence value associated with the first set of the second sensor data;
assign a first weight to the first set of the second sensor data based on the first confidence value;
determine, from the first sensor data, a second confidence value associated with the second set of the second sensor data; and
assign a second weight to the second set of the second sensor data based on the second confidence value.

24. The system of claim 21, wherein the one or more processors are configured to:
activate a first indicator responsive to determining, from the first sensor data, that the distance between the one or more sensors and the measurement site satisfies the threshold, the first indicator indicating to the user that the one or more sensors are positioned correctly relative to the measurement site; and
activate a second indicator responsive to determining, from the first sensor data, that the distance between the one or more sensors and the measurement site does not satisfy the threshold, the second indicator indicating to the user that the one or more sensors are positioned incorrectly relative to the measurement site.

25. The system of claim 21, wherein the one or more processors are configured to activate an alarm responsive to determining, from the first sensor data, that the distance between the one or more sensors and the measurement site does not satisfy the threshold, the alarm indicating to the user that the one or more sensors are positioned incorrectly relative to the measurement site.

26. The system of claim 21, wherein the one or more processors are configured to activate an indicator upon an initial application of the one or more sensors to the measurement site to indicate to the user whether the one or more sensors are positioned correctly relative to the measurement site.

27. The system of claim 21, wherein the one or more processors are configured to:
generate, from the first sensor data, instructions for moving the one or more sensors relative to the measurement site; and
output the instructions for presentation to the user.

28. The system of claim 21, wherein the one or more processors are configured to output the estimated value for presentation to the user while the one or more sensors generate the first sensor data and the second sensor data.

29. The system of claim 21, wherein the physiological parameter comprises an oxygen saturation.

30. The system of claim 21, wherein the physiological parameter comprises a pulse rate.

31. The system of claim 21, wherein the physiological parameter comprises a respiration rate.

32. The system of claim 21, wherein the one or more sensors comprises an optical emitter and an optical detector.

33. The system of claim 21, wherein the one or more sensors are configured to generate the first sensor data and the second sensor data from detected optical radiation.

34. The system of claim 21, wherein the one or more sensors comprises a proximity sensor configured to generate the first sensor data and a physiological sensor configured to generate the second sensor data.

35. The system of claim 34, wherein the proximity sensor comprises a non-optical sensor, and the physiological sensor comprises an optical sensor.

36. The system of claim 21, wherein the one or more sensors comprises an optical sensor configured to generate the first sensor data.

37. The system of claim 21, wherein the one or more sensors comprises a capacitive sensor configured to generate the first sensor data.

38. The system of claim 21, further comprising a display configured to present the estimated value to the user.

39. A computer-implemented method for measuring a physiological parameter comprising:
under control of one or more processors:
accessing first sensor data and second sensor data generated by one or more sensors, the first sensor data being indicative of a distance between the one or more sensors and a measurement site, the second sensor data being usable to determine one or more estimated values of a physiological parameter;
determining, from the first sensor data, that a first set of the second sensor data is generated when the distance between the one or more sensors and the measurement site satisfies a threshold;
determining, from the first sensor data, that a second set of the second sensor data is generated when the distance between the one or more sensors and the measurement site does not satisfy the threshold;
determining, from the first set of the second sensor data and the second set of the second sensor data, an estimated value of the physiological parameter by weighting the first set of the second sensor data differently from the second set of the second sensor data; and
outputting the estimated value for presentation to a user.

40. Non-transitory computer-readable media including computer-executable instructions that, when executed by a computing system, cause the computing system to perform operations comprising:
accessing first sensor data and second sensor data generated by one or more sensors, the first sensor data being indicative of a distance between the one or more sensors and a measurement site, the second sensor data being usable to determine one or more estimated values of a physiological parameter;
determining, from the first sensor data, that a first set of the second sensor data is generated when the distance between the one or more sensors and the measurement site satisfies a threshold;
determining, from the first sensor data, that a second set of the second sensor data is generated when the distance between the one or more sensors and the measurement site does not satisfy the threshold;
determining, from the first set of the second sensor data and the second set of the second sensor data, an estimated value of the physiological parameter by weighting the first set of the second sensor data differently from the second set of the second sensor data; and
outputting the estimated value for presentation to a user.
